(19) United States
(12) Patent Application Publication
(10) Pub. No.: US 2016/0089319 A1
(43) Pub. Date: Mar. 31, 2016

(54) ACTIVES FOR STIMULATING DIFFERENTIATION OF KERATINOCYTES TO LIGHTEN HYPERPIGMENTED SKIN

(71) Applicant: ELC MANAGEMENT LLC, Melville, NY (US)

(72) Inventors: Caroline Françoise POLLEFLIET, Borgerhout (BE); Hugo A.L. CORSTJENS, Maaseik (BE); Lieve DECLERCQ, Ekeren (BE); Daniel B. YAROSH, Merrick, NY (US)

(21) Appl. No.: 14/865,432

(22) Filed: Sep. 25, 2015

Related U.S. Application Data
(60) Provisional application No. 62/056,664, filed on Sep. 29, 2014.

(51) Int. Cl. A61K 8/49; A61Q 19/02; A61K 8/34; A61K 49/00

(52) U.S. Cl. CPC A61K 8/498; A61K 49/0004; A61Q 19/02; A61K 8/347; A61K 8/4973; A61K 8/342

(57) ABSTRACT

A method is provided for identifying a material having an efficacy for reducing color contrast between a hyperpigmented skin lesion and skin surrounding the hyperpigmented skin lesion without directly affecting melanocytes or melanogenesis. The method includes the steps of applying a composition containing a material to be tested to the hyperpigmented skin lesion; and after an interval of time, observing whether the composition has effected at least one of inhibiting proliferation of keratinocytes, stimulating differentiation of keratinocytes, and improving compressive deformation of dermal papillae, in a basal layer of epidermis in the hyperpigmented lesion of skin.

### BACKGROUND

1. Field of Prior Art

The present invention relates to a model for identifying materials which inhibit the proliferation, and stimulate the differentiation, of keratinocytes in hyperpigmented lesions of the skin. Such materials reduce the color contrast between the hyperpigmented lesion and the surrounding skin, thereby evening skin tone.

2. Description of Prior Art

A global concern of consumers, especially women, is unevenness in skin tone. Certain skin cells, melanocytes, are responsible for producing pigment which determines the color of skin. Melanocytes produce and discharge melanin pigment to surrounding keratinocytes in the epidermis. Keratinocytes are driven upwards toward the skin surface by the natural process of cell renewal.

As skin ages, it undergoes transformations which directly influence its appearance. The effect of environmental factors, primarily exposure to ultraviolet (UV) radiation from the sun, and hormonal factors, which result in thinning skin and a reduction in the rate of cellular renewal, can lead to uneven skin tone. Exposure to UV radiation, which has been shown to cause DNA damage, also affects the distribution of melanin in skin. UV radiation can produce reactive oxygen species (ROS) which stimulate melanin production by activating Tyrosinase and other enzymes in melanocytes. Additionally, with a slowing down of cellular renewal, keratinocytes and melanocytes remain in contact longer. As a result of this increased contact time, the amount of melanin transferred to keratinocytes increases. As the epidermis loses its thickness, these melanocytes and keratinocytes, containing increased quantities of dark pigment, are closer to the skin's surface and are therefore more visible.

Solar lentigines or age spots are flat, brown, benign skin lesions which typically occur in aging skin, particularly on the upper surfaces of the hands, face and forearms. The appearance of these lesions is associated with cumulative and intermittent UV exposure and is considered a clinical marker for photodamage of the skin. Solar lentigines are also associated with exposure to particulate levels in polluted air. These lesions are common in both Caucasian and Asian populations but appear to occur earlier and be more pronounced in the skins of Asian individuals.

Histological studies have shown that lesional skin is characterized by hyperpigmentation of the basal layer of the epidermis (i.e., the lowermost layer of the epidermis, adjacent the dermis, containing melanocytes and keratinocytes), an increased number of melanocytes, and the lengthening of the dermal papillae which superficially project into the epidermal region, interlocking with adjacent downward projections of the epidermis (Mehregan, A. H., Lentigo senilis and its evolutions. Invest Dermatol. 1975; 65(5): 429-433; Montagna, W. et al. A reinvestigation of solar lentigines. Arch Dermatol 1980; 116: 1151-1154). It has been suggested that these complex structural modifications may be the result of an increased epidermal proliferation rate and a perturbation of the dermal-epidermal junction (Noblesse, E., et al. Ultrastructure in senile lentigo. Skin Pharmaco. Physiol. 2006; 19: 95-100). It has also been indicated that the release of keratinocyte growth factor and interleukin-1α contribute to the hyperpigmentation of solar lentigines (Chen, N. et al. The role of keratinocyte growth factor in melanogenesis: a possible mechanism for the initiation of solar lentigines. Exp. Dermatol. 19(10), 865-872, 2010). Analysis of gene expression within the lentigo also suggests upregulation of genes associated with a microinflammatory response (Goyarts, E. et al. Morphological Changes Associated with Aging: Age Spots and the Microinflammatory Model of Skin Aging. Ann. N.Y Acad. Sci. 1119:32-39, 2007). This is consistent with the hyperproliferation of keratinocytes observed within the lentigo as part of an inflammatory response.

Clinical research by the inventors on age spots of Caucasian women has shown a higher epidermal proliferation rate associated with these spots compared with adjacent skin. Reflectance Confocal Microscopy (RCM) revealed a profound structural deformation of the dermal papillae, as the alignment pattern of hyperrefractive basal cells shifted from a circle (associated with non-lesional skin) to an irregular non-circular shape (typical of solar lentigines). Additionally, a rise in the number of dermal papillae was observed. It was also shown, in a study conducted over a five year period, that, over time, the solar lentigines became darker and enlarged, and the dermal papillae became more deformed. (Pollefliet, C. et al. Morphological characterization of solar lentigines by in vivo reflectance confocal microscopy: a longitudinal approach. Int J Cosmet Sci. April: 35(2):149-55, 2013).

Uneven pigmentation is a concern for many people, and more particularly, for woman. Age spots, specifically, are considered by most persons to be visually unattractive. The unevenness of skin tone, and especially the appearance of hyperpigmented regions or dark (age) spots in the skin, has led to a multitude of skin lightening agents on the market. Foundation and concealer are frequently used to cover or to camouflage differences in skin tone. Additionally, cosmetic treatments which affect the melanogenesis cascade, particularly those which inhibit the enzyme Tyrosinase, have been shown to decrease the amount of melanin present in solar lentigines, resulting in a lightened pigmented spot. Such agents include, but are not limited to, hydroquinone, which is said to be cytotoxic to melanocytes, and its derivatives, including arbutin; Tyrosinase inhibitors, such as Vitamin C (ascorbic acid) and its derivatives; kojic acid; polyphenols; benzaldehyde and benzoate derivatives; and retinoids, which are derivatives of Vitamin A.

Notwithstanding the overall safety of hydroquinone, its potential adverse effects, including erythema, skin irritation, contact dermatitis, and hypopigmentation of surrounding skin, have stimulated interest in finding other, safer skin lighteners.

The present discovery by the inventors is that materials which inhibit proliferation of keratinocytes and stimulate their differentiation surprisingly and unexpectedly also improve the appearance of age spots and make the skin look more even-toned. More surprisingly, these materials are not classic skin whitening actives (i.e., those actives which are known to affect the melanogenesis cascade).

### SUMMARY OF THE INVENTION

The present invention provides a model for identifying new whitening agents which promote evenness of skin tone. Formulations containing these new whitening agents perform as well as classical whitening formulations to reduce color contrast between age spots and skin surrounding the age spots without the potential adverse effects attributable to traditional whitening actives.

In accordance with the invention, there is provided a method of identifying a material having an efficacy for reducing color contrast between a hyperpigmented skin lesion and skin surrounding the hyperpigmented skin lesion without directly affecting melanocytes or melanogenesis, said method comprising:

(a) applying a composition containing a material to be tested to the hyperpigmented skin lesion; and

(b) after an interval of time, for example, after at least about one months, such as two, three or four months, observing whether the composition has effected at least one of inhibiting proliferation of keratinocytes, stimulating differentiation of keratinocytes, and improving compressive deformation of dermal papillae, in a basal layer of epidermis in the hyperpigmented lesion of skin.

Preferably, the efficacy of the test formulation for improving at least one of inhibiting proliferation of keratinocytes, stimulating differentiation of keratinocytes, and improving compressive deformation of dermal papillae, in a basal layer of epidermis in the hyperpigmented lesion of skin, is evaluated using Reflectance Confocal Microscopy (RCM).

Also provided is a method for lightening skin, comprising applying to skin in need of said lightening, a topical composition comprising at least one active which effects at least one of inhibiting proliferation of keratinocytes, stimulating differentiation of keratinocytes, and improving compressive deformation of dermal papillae in a basal layer of epidermis in a hyperpigmented lesion of skin, wherein the active enables lightening of the skin in a hyperpigmented lesion relative to skin surrounding the hyperpigmented lesion without directly affecting melanocytes or melanogenesis. In some embodiments, the topical composition further comprises a skin lightening active which directly affects melanocytes or melanogenesis.

The invention also concerns an improved topical cosmetic composition for reducing color contrast between a hyperpigmented skin lesion and skin surrounding the hyperpigmented lesion, said composition comprising at least one first active which directly affects melanocytes or melanogenesis, in a cosmetically acceptable vehicle therefor, wherein the improvement comprises including in said composition a second active for effecting at least one of inhibiting proliferation of keratinocytes, stimulating differentiation of keratinocytes, and decreasing compressive deformation of dermal papillae in a basal layer of epidermis in the hyperpigmented lesion of the skin, without directly affecting melanocytes or melanogenesis.

The invention further concerns a regimen for reducing color contrast between a hyperpigmented lesion of skin and skin surrounding the hyperpigmented lesion, comprising:
(a) applying to skin in need of such reduction in color contrast, a first cosmetic composition comprising a first skin lightening active for at least one of inhibiting proliferation of keratinocytes, stimulating differentiation of keratinocytes, and improving compressive deformation of dermal papillae in a basal layer of epidermis in a hyperpigmented lesion of skin, without directly affecting melanocytes or melanogenesis, in a cosmetically acceptable vehicle therefor; and
(b) applying to the skin in need of such reduction in color contrast a second cosmetic composition comprising a second skin lightening active which directly affects melanocytes or melanogenesis, in a cosmetically acceptable vehicle therefor; wherein (a) and (b) may occur in any order.

### DESCRIPTION OF THE FIGURES

FIG. 1A is a bar graph representing the circularity index of dermal papillary rings in hyperpigmented lesions at baseline and after a treatment period of four months with differentiation stimulating formulations containing Phytofix but no traditional whitening actives.

FIG. 1B is a bar graph representing the circularity index of dermal papillary rings in hyperpigmented lesions at baseline and after a treatment period of four months with formulations not containing Phytofix or traditional whitening actives.

FIG. 1C is a bar graph showing differences in the circularity indices shown in FIGS. 1a and 1b.

FIG. 2A is a bar graph indicating the number of dermal papillae in hyperpigmented lesions treated with Phytofix-containing differentiation stimulating formulations.

FIG. 2B is a bar graph showing the number of dermal papillae in hyperpigmented lesions treated with formulations which did not contain Phytofix or traditional whitening actives.

FIG. 2C is a bar graph showing difference observed in the number of dermal papillae shown in FIGS. 2A and 2B.

FIG. 3 is a graph representing color contrast data between the skin of a solar lentigo and surrounding skin after 1, 2, 3 and 4 months after skin treatment.

FIG. 4A is a set of dermoscopic pictures of solar lentigines treated with a differentiation stimulating formulation of the invention.

FIG. 4B is a set of dermoscopic pictures of solar lentigines treated with a differentiation stimulating formulation of the invention followed by a formulation containing conventional whitening actives.

FIG. 5 is a set of bar graphs showing the effect on circularity index of papillary rings in solar lentigines treated with a differentiation stimulating formulation of the invention or the differentiation stimulating formulation followed by a formulation containing conventional whitening actives compared with treatment with a vehicle/base for the differentiation stimulating formulation.

FIG. 6 is a set of dermoscopic pictures of solar lentigines and Reflectance Confocal Microscopy (RCM) images of the solar lentigines recorded at the dermal-epidermal interface representing the improvement in circularity index after treatment with a differentiation stimulating formulation of the invention.

FIG. 7 is a set of bar graphs representing the number of dermal papillae present in solar lentigines treated with a differentiation stimulating formulation of the invention or the differentiation stimulating formulation followed by a formulation containing conventional whitening actives compared with treatment with a vehicle/base for the differentiation stimulating formulation.

FIG. 8 is a set of dermoscopic pictures of solar lentigines and RCM images of the solar lentigines recorded at the dermal-epidermal interface representing the improvement in number of dermal papillary rings after treatment with a differentiation stimulating formulation of the invention.

FIG. 9A is a set of bar graphs showing the effect on epidermal thickness of solar lentigines treated with a differentiation stimulating formulation of the invention or the differentiation stimulating formulation followed by a formulation containing conventional whitening actives compared with treatment with a vehicle/base for the differentiation stimulating formulation.

FIG. 9B is a set of bar graphs showing the effect on thickness of the stratum corneum of solar lentigines treated with a differentiation stimulating formulation of the invention or the differentiation stimulating formulation followed by a formulation containing conventional whitening actives compared with treatment with a vehicle/base for the differentiation stimulating formulation.

FIG. 10 is a set of bar graphs showing epidermal proliferation as measured by auto-fluorescence of solar lentigines treated with a differentiation stimulating formulation of the invention or the differentiation stimulating formulation followed by a formulation containing conventional whitening actives compared with treatment with a vehicle/base for the differentiation stimulating formulation.

### DETAILED DESCRIPTION OF THE PREFERRED EMBODIMENTS OF THE INVENTION

Solar lentigo is commonly observed in photoaged skin. It is characterized by solar lentigines or hyperpigmented age spots or lesions appearing in chronically irradiated skin. These spots are benign and typically occur in the skins of individuals after age 50. From microscopic studies, it is known that there are significant modifications of lesional skin in comparison with adjacent normal skin, including a hyperpigmented basal layer with melanin accumulation and decrease in evacuation of melanin overproduction, elongation of epidermis rete ridges, and disorganization and disruption of dermal epidermal junction (reduced barrier quality) associated with an increase of keratinocyte basal microvillosity.

Previous investigations by the inventors have shown that these lesions demonstrate increased Transepidermal Water Loss (TEWL) and decreased caspase-14 activity, supporting the observations of others that age spots are characterized by an impaired barrier function. Investigations by the inventors, using RCM, confirmed profound structural deformations of the dermal papillae in solar lentigines, and led them to consider whether the deformations might be reversible. The capabilities of various topical cosmetic formulations to improve or reverse the deformations were investigated as discussed below.

### EXAMPLES

### 1. Effect of Cosmetic Raw Materials on the Morphology of Solar Lentigenes

Forty-one volunteers were enrolled in a four month clinical study. Only panelists with clear visible age spots on their hand could participate. The mean age of the panelists was 61 years with the minimum and maximum ages being 47 and 74, respectively. All volunteers were Caucasian and exhibited Fitzpatrick skin type II or III. The test area was a solar lentigo on the dorsal side of each of both hands. Panelists with any kind of dermatological disorder or hypersensitivity or allergy to ethanol and/or cosmetic products were excluded from the study. Written informed consent was obtained from each volunteer before entrance into the study.

Phytofix, an active containing sphingolipids, triglycerides and sterols which are derived from sunflower, barley and cucumber extracts, is said to mimic the structure of the skin's membrane and is widely used for its skin barrier repair properties. Various formulations, containing no traditional whitening actives, were evaluated for their age spot lightening efficacy. Formulations 1-3, containing Phytofix, and formulations 4 and 5, without Phytofix, are shown in Table 1, below.

Formulation 1 was tested on 12 panelists (one age spot/panelist for a total of 12 age spots). Formulation 2 was applied by 13 panelists (one age spot/panelist for a total of 13 age spots), Formula 3 was used by 16 panelists (two age spot/panelist for a total of 32 age spots), Formulation 4 was tested on 14 panelists (one age spot/panelist for a total of 14 age spots) and formulation 5 was used by 10 panelists (one age spot/panelist for a total of 10 age spots). Each panelist liberally applied the formulation, twice a day (morning and evening) for 4 months, on the entire dorsal surface of her hand. Panelists were instructed not to use any other cosmetic products on their hands during the clinical study. One age spot on each hand, selected by the investigator, was evaluated monthly.

TABLE 1

| | Formula | | | | |
|---|---|---|---|---|---|
| | 1 | 2 | 3 | 4 | 5 |
| INGREDIENT NAME | WEIGHT PERCENT | | | | |
| GRANSIL TMG-5 JH GEL | 23.000000 | | | 23.000000 | 23.000000 |
| XIAMETER AFE-0100 AF EMULSION FG | | 0.000194 | 0.000194 | | |
| FLORALOZONE | | 0.006000 | 0.006000 | | |
| BHT | | 0.010000 | 0.010000 | | |
| CORIANDER OIL A | | 0.020000 | 0.020000 | | |
| MALT EXTRACT BROTH 214912 | | 0.029410 | 0.029410 | | |
| YEAST EXTRACT SC | | | | | |
| GRANSIL IDS GEL | 10.000000 | | | 10.000000 | 10.000000 |
| 1,3 BUTYLENE GLYCOL | 6.007000 | | | 6.007000 | 6.007000 |
| PERMETHYL 99A | 2.000000 | | | 2.000000 | 2.000000 |
| SIMULGEL 600 | 1.500000 | | | 1.500000 | 1.500000 |
| CARBOWAX 300 SENTRY | 1.000000 | | | 1.000000 | 1.000000 |
| ARIST OFLEX AVC | 0.600000 | | | 0.600000 | 0.600000 |
| PHYT OFIX | 0.500000 | 0.500000 | 0.500000 | | |
| TWEEN 20 L | 0.500000 | | | 0.500000 | 0.500000 |
| BARGUARD CP/DIOCIDE | 0.300000 | | | 0.300000 | 0.300000 |
| VITAMIN E, USP, FCC, CODE 0420085 | 0.200000 | | | | |
| SODIUM RIBONUCLEIC ACID | 0.100000 | 0.100000 | 0.100000 | | |
| FD&C YELLOW NO. 5, 08005 | 0.000260 | | | 0.000260 | 0.000260 |
| FD&C YELLOW NO. 6, 08006 | 0.000090 | | | 0.000090 | 0.000090 |
| XIAMETER PMX-200 SILICONE FL. 10CS | | 10.000000 | 10.000000 | | |
| XIAMETER PMX-200 SILICONE FL 100CS | | 5.000000 | 5.000000 | | |
| SEPIGEL 305 | | 3.400000 | 3.400000 | | |
| CATEZOMES SA-20 JPN | | 2.500000 | 2.500000 | | |
| TWEEN 40 LQ | | 2.500000 | 2.500000 | | |
| HYDROLITE 5, 2/016020 | | 2.000000 | 2.000000 | | |
| MYRISTYL ALCOHOL NACOL 14-98 | | 1.000000 | 1.000000 | | |
| SUCROSE, ULTRA PURE | | 0.750000 | 0.750000 | | |
| RHODASURF L-790 | | 0.750000 | 0.750000 | | |
| BRONIDOX 1160 | | 0.700000 | 0.700000 | | |
| CORN OIL N.F. REFINED | | 0.500000 | 0.500000 | | |
| SORBITOL SOLUTION 70% | | 0.500000 | 0.500000 | | |
| BIO-CONVERTED WHITE BIRCH/E.L. | | 0.500000 | 0.500000 | | |
| CAUSTIC SODA 30% | | 0.197000 | 0.197000 | | |
| N-ACETYL-D-GLUCOSAMINE | | 0.194070 | 0.194070 | | |
| GERMAZIDE C R10284 | | 0.100000 | 0.100000 | | |
| GRASNOW AE | | 0.100000 | 0.100000 | | |
| HEDIONE 964898 | | 0.080000 | 0.080000 | | |
| GRAPEFRUIT CALIFORNIA | | 0.060000 | 0.060000 | | |
| CITRIC ACID-ANHYD., USP-FCC (GRANU.) | | 0.050000 | 0.050000 | | |
| LAVENDER OIL SPECIAL 40/42 | | 0.034000 | 0.034000 | | |
| EDETA BD/NA2 | 0.050000 | 0.020000 | 0.020000 | 0.050000 | 0.050000 |
| DM-FLUID A-6CS | 5.000000 | 3.000000 | 3.000000 | 5.000000 | 5.000000 |
| HYALURONIC ACID, SODIUM SALT, POWD | 0.100000 | 0.100000 | 0.100000 | 0.100000 | 0.100000 |
| KF-6017 | 1.000000 | 0.750000 | 0.750000 | 1.500000 | 1.000000 |
| PHENOXET OL | 0.290000 | 0.100000 | 0.100000 | 0.290000 | 0.290000 |
| DEIONIZED WATER | 47.852650 | 64.449326 | 64.449326 | 48.152650 | 48.652650 |
| | 100.000000 | 100.000000 | 100.000000 | 100.000000 | 100.000000 |

Formula 1 is prepared as follows:

Prepare premix B (sequence 10) at the beginning of the batch by heating 3% Butylene glycol to 70° C. in a vessel, adding 1% Carbowax, and mixing at 2000 RPM.

In a separate support kettle:
Sequence 1: Add 10% Hyaluronic acid to support kettle and side sweep at 8 RPM.
Sequence 2: Add 0.5% Tween 20 to support kettle.
Sequence 3: Add 23% Gransil TMG-5 to support kettle and mix at 2500 RPM,
add 10% Gransil IDS to support kettle,
add 5% DM-fluid to support kettle,
add 2% Permethyl to support kettle,
add 0.5% Phytofix to support kettle, and
adjust temperature to 25° C.
Sequence 4: Add 1% KF-6017 to support kettle, and mix until uniform and smooth.
Sequence 5: Add 38% deionized water to main kettle,
heat at 50°,
mix at 531 RPM,
side weep at 5 RPM,
add 0.05% EDETA to main kettle,
add 0.1% Sodium ribonucleic acid to main kettle,
adjust temperature to 25° C.
Sequence 6: Add 0.6% Aristoflex (50-3214) to main kettle.
Prepare premix A (sequence 9) by introducing 3% Butylene glycol into a vessel, heating at 70° C., then adding 0.3% Barguard cp.
Sequence 7: Add 0.2% Phenoxetol to main kettle, and introduce batch from support kettle.
Sequence 8: Add 0.2% Vitamin E to main kettle.
Sequence 9: Add premix A to main kettle.
Sequence 10: Add premix B to main kettle.
Sequence 11: Add 1.5% Simulgel 600 to main kettle.
Sequence 12: Add 0.026% FD&C Yellow no 5 to main kettle, add 0.009% FD&C Yellow no 6 to main kettle, and mix until uniform.

Formula 2 is prepared as follows:

Prepare premix B (sequence 3) by adding 2.5% Tween 40 to a vessel, mixing at 3000 RPM, adding 0.5% Phytofix, heating to 80° C., and then turning the heat off.

Prepare premix C (sequence 5) by adding 5% deionized water to a separate vessel, mixing at 2500 RPM, and adding 0.1% Ribonucleic acid.

Prepare premix D (sequence 6) by adding 2% Hydrolite to a separate vessel, mixing at 2000 RPM, heating the vessel to 40° C., and adding 0.7% Bronidox.
Sequence 1: Add 47.87% deionized water to the main kettle,
adjust temperature to 60° C., then side sweep on 12 RPM, and mix at 910 RPM,
add 0.02% Edeta to the main kettle,
add 0.75% Sucrose to the main kettle,
add 10% Hyaluronic acid to the main kettle,
add 0.1% Germazide to the main kettle,
add 0.5% Sorbitol to the main kettle,
add 0.75% Rhodasurf to the main kettle, and
add 0.05% Citric acid to the main kettle,
Sequence 2: Add 5% Xiameter to support kettle,
mix at 1000 RPM,
heat at 60° C.,
add 10% Xiameter to support kettle,
add 3% DM-fluid to support kettle,
add 1% Myristyl alcohol to support kettle,
add 0.5% Corn oil to support kettle,
add 0.75% KF-6017 to support kettle, and
add 0.01% BHT to support kettle.
Sequence 3: Add premix B to the main kettle, then add batch from support kettle.
Sequence 4: Cool main kettle on 30° C. setting. When temperature reaches 40° C., add 0.1% Grasnow AE to the main kettle,
add 2.5% Catezomes to the main kettle,
add 2% Yeast extract to the main kettle,
add 0.5% Bio-converted white birch to the main kettle, mix at 1220 RPM then side sweep at 15 RPM.
Sequence 5: Add premix C to the main kettle.
Sequence 6: Add premix D to the main kettle.
Sequence 7: Add 0.2% Maskent blend to the main kettle.
Sequence 8: Adjust pH to 5 by adding 0.197% Caustic soda to the main kettle.
Sequence 9: Add 3.4% Sepigel 305 to the main kettle.

Formula 3 is prepared as follows:

Prepare premix B (sequence 3) by adding 2.5% Tween 40 to a vessel, mixing at 3000 RPM, adding 0.5% Phytofix, heating to 80° C., and then turning the heat off.

Prepare premix C (sequence 6) by adding 5% deionized water to a separate vessel, mixing at 2500 RPM, and adding 0.1% Ribonucleic acid.

Prepare premix D (sequence 8) by adding 2% Hydrolite to a separate vessel, mixing at 2000 RPM, heating to 40° C., and adding 0.7% Bronidox.
Sequence 1: Add 47.87% deionized water to the main kettle,
adjust temperature to 60° C.,
side sweep at 12 RPM,
mix at 910 RPM,
add 0.02% Edeta to the main kettle
add 0.75% Sucrose to the main kettle,
add 10% Hyaluronic acid to the main kettle,
add 0.1% Germazide to the main kettle,
add 0.5% Sorbitol to the main kettle,
add 0.75% Rhodasurf to the main kettle, and
add 0.05% Citric acid to the main kettle.
Sequence 2: Add 5% Xiameter (68-0451) to support kettle;
mix at 1000 RPM,
heat to 60° C.,
add 10% Xiameter to support kettle
add 3% DM-fluid to support kettle,
add 1% Myristyl alcohol to support kettle,
add 0.5% Corn oil to support kettle,
add 0.75% KF-6017 (60-0031) to support kettle, and
add 0.01% BHT (51-0669) to support kettle.
Sequence 3: Add premix B to the main kettle, and add the contents of the support kettle.
Sequence 4: Switch heat off, and cool main kettle on 30° C. setting. When temperature reaches 40° C., add 0.1% Grasnow AE to the main kettle,
add 2.5% Catezomes to the main kettle,
add 2% Yeast extract to the main kettle,
add 0.5% Bio-converted white birch to the main kettle,
mix at 1220 RPM then side sweep speed at 15 RPM.
Sequence 5: Add premix C to the main kettle.
Sequence 6: Add premix D to the main kettle.
Sequence 7: Add 0.2% Maskent blend to the main kettle.
Sequence 8: Adjust pH to 5 by adding 0.197% Caustic soda to the main kettle.
Sequence 9: Add 3.4% Sepigel 305 to the main kettle.

Formula 4 is prepared as follows:
Sequence 1: Add 10% Hyaluronic acid to support kettle and side sweep on 8 RPM.
Sequence 2: Add 0.5% Tween 20 to support kettle.
Sequence 3: Add 23% Gransil TMG-5 to support kettle,
mix at 2500 RPM,
add 10% Gransil IDS to support kettle,
add 5% DM-fluid to support kettle,
add 2% Permethyl to support kettle, and
adjust temperature to 25° C.
Sequence 4: Add 1.5% KF-6017 to support kettle and mix until uniform and smooth.
Sequence 5: Add 38% deionized water to main kettle,
heat on 50° C.,
mix at 481 RPM,
side sweep on 5 RPM,
add 0.05% EDETA to main kettle, and
turn heat off.
Sequence 6: Cool main kettle to 25° C. When temperature reaches 25° C., add 0.6% Aristoflex to main kettle.

Prepare premix A (sequence 8) by adding 3% Butylene glycol to a vessel and heating to 70° C., adding 0.3% Barguard cp, then adding 1% Carbowax 30, and mixing at 2000 RPM.
Sequence 7: Add 0.2% Phenoxetol to main kettle. Add ingredients from support kettle to main kettle.
Sequence 8: Add premix A to main kettle.
Sequence 9: Add 1.5% Simulgel 600 to main kettle.
Sequence 10: Add 0.026% FD&C Yellow no. 5 to main kettle,
add 0.009% FD&C Yellow no. 6 to main kettle, and mix until uniform.

Formula 5 is prepared as follows:
Sequence 1: Add 10% Hyaluronic acid to support kettle and side sweep on 8 RPM.
Sequence 2: Add 0.5% Tween 20 to support kettle.
Sequence 3: Add 23% Gransil TMG-5 to support kettle,
mix at 2500 RPM,
add 10% Gransil IDS to support kettle,
add 5% DM-fluid to support kettle,
add 2% Permethyl to support kettle, and adjust temperature to 25° C.
Sequence 4: Add 1.5% KF-6017 to support kettle and mix until uniform and smooth.
Sequence 5: Add 38% deionized water to main kettle,
heat main kettle to 50° C.,
mix at 481 RPM, then side sweep on 5 RPM,
add 0.05% EDETA to main kettle, and turn heat off.
Sequence 6: Cool main kettle to 25° C., and when temperature reaches 25° C., add 0.6% Aristoflex to main kettle.

Prepare premix A (sequence 8) by adding 3% Butylene glycol to a vessel and heating at 70° C., then adding 0.3% Barguard cp, adding 1% Carbowax 30, and mixing at 2000 RPM.
Sequence 7: Add 0.2% Phenoxetol to main kettle; then add contents of support kettle to main kettle.
Sequence 8: Add premix A to main kettle.
Sequence 9: Add 1.5% Simulgel 600 to main kettle.
Sequence 10: Add 0.026% FD&C Yellow no. 5 to main kettle,
add 0.009% FD&C Yellow no. 6 to main kettle, and mix until uniform.

In this study, the effect of cosmetic raw materials on the morphology of solar lentigines was evaluated by Reflectance Confocal Microscopy (RCM) using a Vivascope (Lucid 1500) before treatment and then after 4 months of treatment. RCM is a noninvasive technique for “in vivo” examination of the skin. The Vivascope is placed on the dorsal side of the hand. An ultrasound gel (refractive index 1.36) is used as immersion medium between the objective lens and the skin. RCM acquires horizontal tissue images of a field of view of 500×500 μm and has an automated stepper obtaining sequentially deeper individual images, 3 μm apart, from the corneal layer to the superficial dermis (Z-axis stack) at the same point on the horizontal plane (XY-axis). From each age spot, six optical sections of the skin were obtained from the corneal layer to the superficial dermis.

In a confocal microscope, near-infrared light from a diode laser is focused on a microscopic skin target. As this light passes between cellular structures having different refractive indices, it is naturally reflected, and this reflected light is then captured and recomposed into a two-dimensional gray scale image by computer software. Focusing the microscope (that is, adjusting the focal point on the z-axis) allows images of different levels within the skin to be obtained. Detection requires the presence of intrinsic microstructure-specific contrast, which is provided by melanin, hemoglobin and cellular organelles. Melanin is the best endogenous contrast agent in pigmented skin, allowing the detection of melanin-containing cells due to its high refractivity. Commercially available microscope systems of this type can create images with sufficient detail to be used in histological analysis (Calzavara-Pinton P., Longo C., Venturini M., Sala R., Pellacani G. Photochem Pholobiol. 2008 November-December; 84(6): 1421-30). The Vivascope 1500 system is a reflectance confocal microscope that has the capability to image cells in real-time, layer by layer through living tissue. These images can be used to measure the thickness of the skin and its different layers and the shape of the dermal papillary rings. The Vivascope 1500 uses a near infrared laser at 830 nm operating at a power of less than 20 mW and is equipped with a 30× objective lens of numerical aperture 0.9.

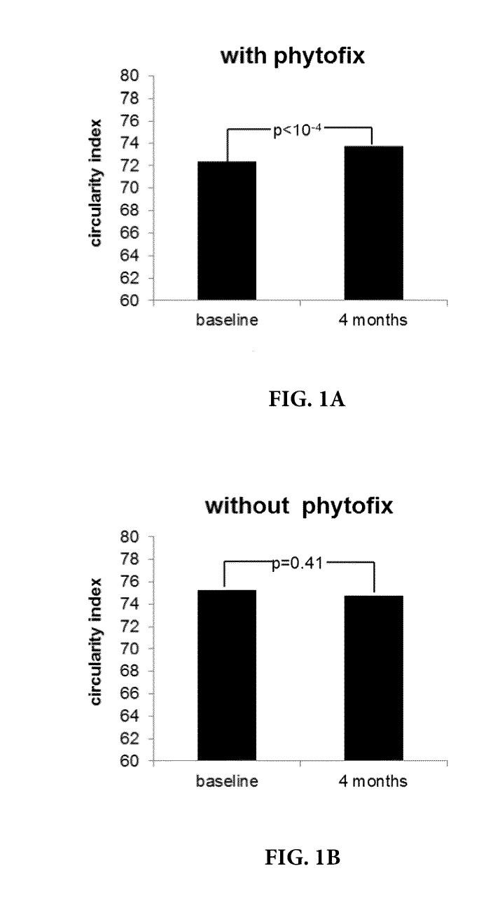

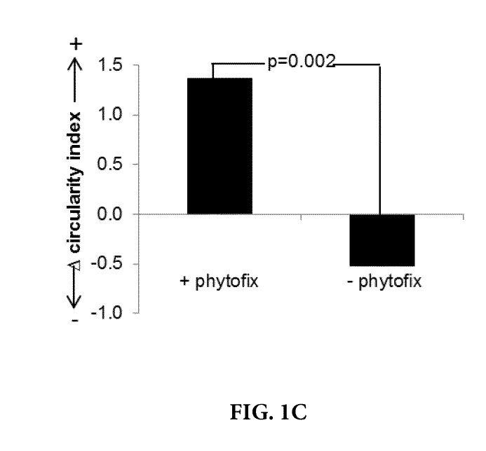

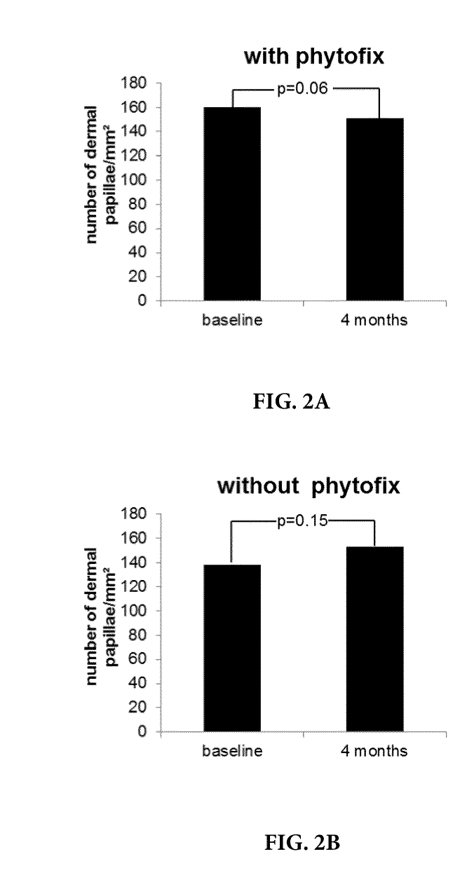

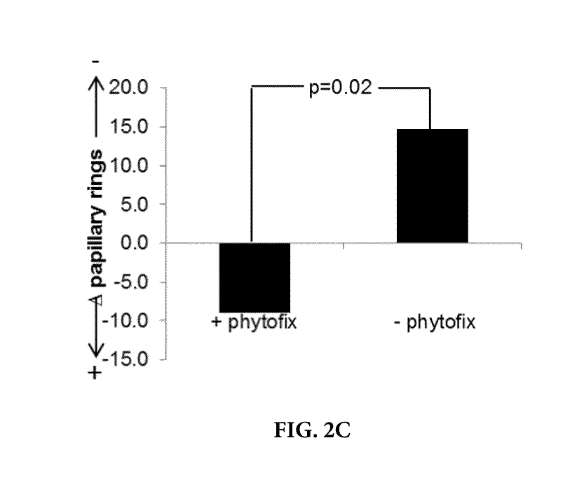

The circularity index and the number of hyperrefractive dermal papillae (per mm²) were calculated from the RCM images. The circularity index of a perfect circle equals 100; therefore, a lower index corresponds to a more pronounced deviation from a circle. As an increased number of dermal papillary rings is also associated with deformed morphology of the lesions, as compared with non-lesional areas of the skin, a decrease in the number of rings is considered to be an improvement in morphology. Time dependent effects were evaluated using a paired Student's t test. Differences between treatments were evaluated using a Student's t test for unpaired samples. The results are represented in FIGS. 1a, b, c; and FIGS. 2a, b, c.

FIG. 1a represents the circularity index at baseline and after a treatment period of four months with formulations with or without Phytofix, and each formulation containing no traditional whitening actives. Treatment with formulations 1, 2 and 3, each containing 0.5% Phytofix, showed a statistically significant increase (p<10⁻⁴) in circularity index (from 72.33 to 73.69) of the dermal papillae of lesions (age spots) indicating an improvement (i.e., reduction) of the deformed morphology of the basement membrane of the hyperpigmented spots. The circularity index of the dermal papillae of non-lesional skin was found to be about 80 (not shown). As indicated in FIG. 1b, a four month treatment with formulations 4 and 5, without Phytofix, showed no effect on the circularity index (which ranged from 75.24 to 74.71).

The differences in the circularity indices observed before and after treatment were calculated for the treatments with or without Phytofix (FIG. 1c). The formulations containing Phytofix showed an increase in circularity index of 1.4, indicating an improvement of the deformed morphology, while for the formulations without Phytofix, there was no improvement (i.e., a decrease of 0.5). The difference between the two sets of formulations was statistically significant, suggesting that Phytofix was contributing to the effects observed.

The inventors had previously observed that, over time (without treatment), the average circularity index in age spots decreases over 5 years by 1.7 (Pollefliet, C. et al. Morphological characterization of solar lentigines by in vivo reflectance confocal microscopy: a longitudinal approach. Int J Cosmet Sci. April: 35(2):149-55, 2013).

As shown in FIG. 2a, the number of dermal papillae in hyperpigmented lesions treated with Phytofix-containing formulations 1, 2 and 3 decreased from 160.7 to 151.7. This phenomenon was not observed for lesions treated with formulations 4 and 5, which did not contain Phytofix, as indicated in FIG. 2b, where the number of dermal papillae increased from 138.7 to 153.3.

The difference in the number of dermal papillae before and after the treatment also was calculated. This difference, as indicated in FIG. 2c, was negative for the Phytofix-containing formulations (−9.0) indicating a reduction in the number of papillary rings. The treatment of lesions with formulations without Phytofix showed an opposite trend (+14.6) with the difference between the treatments reaching statistical significance.

The inventors had previously observed that, over time (without treatment), the average number of dermal papillae in age spots tended to increase over 5 years by 18.7 (Pollefliet, C. et al. Morphological characterization of solar lentigines by in vivo reflectance confocal microscopy: a longitudinal approach. Int J Cosmet Sci. April: 35(2):149-55, 2013).

Previous studies by the inventors indicated that Phytofix, used in formulations in a range of from about 0.001 wt. % to about 2 wt. %, demonstrated efficacy in improving the morphology of the basement membrane in hyperpigmented lesions (results not shown).

It was appreciated that each of the formulations demonstrating positive effects on the morphology of the basement membrane of hyperpigmented lesions also contained sodium ribonucleic acid in addition to Phytofix. However, since no improvement of the morphology of the basement membrane was seen using a formulation containing sodium ribonucleic acid but no Phytofix, the inventors concluded that the positive effects on the morphology were not caused by sodium ribonucleic acid (results not shown).

The inventors also observed that, in some cases, formulations containing a classic whitening active, for example, UP302, (i.e., dimethoxytolyl propyl resorcinol, derived from the Dianella ensifolia plant) or molasses extract, in combination of with Phytofix, resulted in the disappearance of the positive effects on the morphology of the solar lentigines demonstrated by formulations containing Phytofix but no classic whitening actives (results not shown). On the other hand, treatment with certain formulations containing Phytofix with a combination of known whitening actives, including Yeast extract XP, Black strap molasses, Phytoclar II, AS-G (ascorbyl glucoside) powder and Trametes versicolor, or with formulations containing Phytofix with a combination of known whitening actives, including Moms nigra (mulberry) root extract, Scutellaria baicalensis extract and Vitis vinifera (grape) extract, demonstrated improved morphology of the basement membrane (results not shown). However, treatment with formulations containing these known whitening actives in addition to the Phytofix did not result in any greater improvement in morphology compared to the use of formulations containing Phytofix without the known whitening actives.

The data indicate that, generally, topical formulations 1, 2 and 3, containing Phytofix, were able to improve the morphology of the basement membrane in solar lentigines, while formulations 4 and 5, which did not contain Phytofix, were not capable of doing so. Moreover, skin treatments with formulations containing the material Phytofix, in the absence of traditional whitening actives, had positive effects on the deformed basement membrane in the skin of age spots and tended to normalize this deformation. This effect was not observed when treating hyperpigmented lesions with formulations lacking Phytofix. Furthermore, the addition of some traditional whitening actives to Phytofix-containing formulations had no apparent effect on the capability of Phytofix to effect a positive change in the morphology of the basement membrane of age spots, while the combination of other traditional whitening actives in Phytofix-containing formulations appeared to neutralize the positive effect of Phytofix on morphology. There remains a need to investigate the incompatibilities to avoid mixing ingredients in the formulations which may negatively impact the efficacy of the Phytofix to improve the morphology of the basement membrane of hyperpigmented lesions.

### 2. Effect of Differentiation Stimulating Actives on Skin Tone and Morphology of Dermal Papillae in Solar Lentigines

In this study, various formulations (shown in Table 2, below) were evaluated for their capabilities in improving uneven skin tone and/or morphology of dermal papilla in solar lentigines. The solar lentigines of each of three panelist groups were treated with either (1) a differentiation stimulating formula containing the following actives: salicylic acid (anti-proliferative function), resveratrol (Sirt-1 activation function), MPC (migration function), Sclareolide (i.e., Clary Sage fermented extract, titrated in Sclareolide-maturation function) and Phytofix (skin barrier recovery function), or (2) the differentiation stimulating formula followed by a whitening formula containing the following known whitening actives: Yeast extract XP, Trametes Extract, AS-G powder, Phytoclar II; Nivitol UP-302, and Viapure licorice, or (3) with a control formulation, i.e., a base or vehicle for water soluble actives.

TABLE 2

| | Formula | | |
|---|---|---|---|
| | 6 | 7 | 8 |
| INGREDIENT NAME | WEIGHT PERCENT | | |
| RESVERATROL 85% | | 0.100000 | |
| TRIS AMINO ULTRA PC | | 0.200000 | 0.645000 |
| TROMETHAMINE | | | |
| SALICYLIC ACID USP (POWDER) | | 0.250000 | |
| MPC | | 0.500000 | |
| PHYTOFIX | | 1.000000 | |
| CLARY SAGE FERMENTED EXTRACT | | 0.050000 | |
| SIMULGEL 600 | 1.500000 | 1.500000 | 1.500000 |
| ACRYLAMIDE/SODIUM ACRYLOYLDIMETHYLTAURATE | | | |
| COPOLYMER/WATER\AQUA\EAU/ISOHEXADECANE/ | | | |
| POLYSORBATE 80 | | | |
| CARBOWAX 300 SENTRY | 1.000000 | 1.000000 | 1.000000 |
| PEG-6 | | | |
| ARISTOFLEX AVC | 0.600000 | 0.600000 | 0.600000 |
| AMMONIUM ACRYLOYLDIMETHYLTAURATE/VP | | | |
| COPOLYMER | | | |
| TWEEN 20 L | 0.500000 | 0.500000 | 0.500000 |
| POLYSORBATE 20 | | | |
| BARGUARD CP/DIOCIDE | 0.300000 | 0.300000 | 0.300000 |
| CAPRYLYL GLYCOL/PHENOXYETHANOL/HEXYLENE | | | |
| GLYCOL | | | |
| PHENOXETOL | 0.290000 | 0.290000 | 0.300000 |
| PHENOXYETHANOL | | | |
| HYALURONIC ACID, SODIUM SALT, POWD | 0.100000 | 0.100000 | 0.100000 |
| SODIUM HYALURONATE | | | |
| EDETA BD/NA2 | 0.050000 | 0.050000 | 0.050000 |
| DISODIUM EDTA | | | |
| KF-6017 | 1.500000 | 1.500000 | 1.500000 |
| PEG-10 DIMETHICONE | | | |
| PERMETHYL 99A | 2.000000 | 2.000000 | 2.000000 |
| ISODODECANE | | | |
| DM-FLUID A-6CS | 5.000000 | 5.000000 | 5.000000 |
| DIMETHICONE | | | |
| 1,3 BUTYLENE GLYCOL | 6.000000 | 6.000000 | 6.007000 |
| BUTYLENE GLYCOL | | | |
| GRANSIL IDS GEL | 10.000000 | 10.000000 | 10.000000 |
| POLYSILICONE-11/ISODODECANE | | | |
| DEIONIZED WATER | 48.160000 | 46.060000 | 33.063976 |
| PURIFIED WATER | | | |
| GRANSIL DMDM-25 | 23.000000 | 23.000000 | |
| GRANSIL TMG-5 JH GEL | | | 23.000000 |
| CYCLOPENTASILOXANE/DIMETHICONE/POLYSILICONE- | | | |
| 11/CHOLESTEROL/SQUALANE/GLYCYRRHETINIC | | | |
| ACID/TRITICUM VULGARE (WHEAT) GERM | | | |
| EXTRACT/HORDEUM VULGARE (BARLEY) EXTRACT | | | |
| CATEZOMES SA-20 JPN | | | 2.500000 |
| WATER/SALICYLIC ACID/SODIUM HYDROXIDE/BUTYLENE | | | |
| GLYCOL/DI-C12-18 ALKYL DIMONIUM CHLORIDE | | | |
| BIO-CONVERTED WHITE BIRCH/E.L. | | | 0.500000 |
| ECLUSTERED WATER (−)\AQUA\EAU DE-STRUCTUREE (−)/ | | | |
| DECLUSTERED WATER (+)\AQUA\EAU DE-STRUCTUREE | | | |
| (+)/BETULA ALBA (BIRCH) BARK | | | |
| EXTRACT/SACCHAROMYCES LYSATE EXTRACT | | | |
| RICE DEFENSE COMPLEX R | | | 0.500000 |
| BUTYLENE GLYCOL/WATER/ORYZA SATIVA (RICE) BRAN | | | |
| EXTRACT | | | |
| CAFFEINE POWDER | | | 0.200000 |
| CAFFEINE | | | |
| VITAMIN E, USP, FCC, CODE 0420085 | | | 0.200000 |
| TOCOPHERYL ACETATE | | | |
| N-ACETYL-D-GLUCOSAMINE | | | 0.194070 |
| ACETYL GLUCOSAMINE | | | |
| NIVITOL UP-302 | | | 0.180000 |
| DIMETHOXYTOLYL PROPYLRESORCINOL | | | |
| VIAPURE LICORICE/18BGLYCYRRHETINIC | | | 0.100000 |
| GLYCYRRHETINIC ACID | | | |
| SODIUM RIBONUCLEIC ACID | | | 0.100000 |
| SODIUM RIBONUCLEIC ACID | | | |
| TETRAHYDROCURCUMINOIDS | | | 0.100000 |
| CURCUMA LONGA (TURMERIC) ROOT EXTRACT | | | |
| VIAPURE CITRUS | | | 0.030000 |
| CITRUS GRANDIS (GRAPEFRUIT) PEEL EXTRACT | | | |
| MALT EXTRACT BROTH 214912 | | | 0.029410 |
| MALT EXTRACT | | | |
| FD&C YELLOW NO. 5, 08005 | | | 0.000260 |
| YELLOW 5 (CI 19140) | | | |
| XIAMETER AFE-0100 AF EMULSION FG | | | 0.000194 |
| SIMETHICONE | | | |
| FD&C YELLOW NO. 6, 08006 | | | 0.000090 |
| FD&C YELLOW NO. 6 | | | |
| YEAST EXTRACT XP | | | 5.000000 |
| WATER\AQUA\EAU/YEAST EXTRACT\FAEX\EXTRAIT DE | | | |
| LEVURE | | | |
| TRAMETES EXTRACT 140080 | | | 2.000000 |
| WATER\AQUA\EAU/TRAMETES EXTRACT | | | |
| AS-G POWDER | | | 1.800000 |
| ASCORBYL GLUCOSIDE | | | |
| PHYTOCLAR II BG NEXTGEN | | | 1.000000 |
| BUTYLENE GLYCOL/SCUTELLARIA BAICALENSIS ROOT | | | |
| EXTRACT/MORUS BOMBYCIS ROOT EXTRACT | | | |
| | 100.000000 | 100.000000 | 100.000000 |

Formula 6 was prepared as follows:
Sequence 1: Add 10% Hyaluronic acid to support kettle, and side sweep on 8 RPM.
Sequence 2: Add 0.5% Tween 20 to support kettle.
Sequence 3: Add 23% Gransil TMG-5 to support kettle, and mix at 2500 RPM,
add 10% Gransil IDS to support kettle,
add 5% DM-fluid to support kettle,
add 2% Permethyl to support kettle, and adjust temperature to 25° C.
Sequence 4: Add 1.5% KF-6017 to support kettle, and mix until uniform and smooth.
Sequence 5: Add 38.35% deionized water to main kettle, and heat main kettle at a setting of 50° C.,
mix at 481 RPM, then side sweep at 5 RPM,
add 0.05% EDETA to main kettle, and then turn off the heat.
Sequence 6: Cool main kettle to 25° C., add 0.6% Aristoflex to main kettle, and then add contents of support kettle.

Prepare premix A (sequence 7) by adding 3% Butylene glycol to a vessel, heating to
70° C., adding 0.2% Phenoxetol, then adding 0.3% Barguard cp, followed by adding 1% Carbowax 30, and mixing at 2000 RPM.
Sequence 7: aaAdd premix A to main kettle.
Sequence 8: Add 1.5% Simulgel 600 to main kettle and mix until uniform.
Formula 7 was prepared as follows:
Sequence 1: Add 10% Hyaluronic acid to support kettle, and side sweep at 8 RPM.
Sequence 2: Add 0.5% Tween 20 to support kettle.
Sequence 3: Add 23% Gransil TMG-5 to support kettle, and mix at 2500 RPM,
add 10% Gransil IDS to support kettle,
add 5% DM-fluid to support kettle,
add 2% Permethyl to support kettle, and adjust temperature to 25° C.
Sequence 4: Add 1.5% KF-6017 to support kettle, and mix until uniform and smooth.
Sequence 5: Add 35.25% deionized water to main kettle,
heat to 50° C.,
mix at 481 RPM, then side sweep at 5 RPM,
add 0.05% EDETA to main kettle,
add 0.5% MPC to main kettle,
add 1% Phytofix to main kettle, and then turn heat off.
Sequence 6: Cool main kettle to 25° C.; then add 0.6% Aristoflex to main kettle; and then
add contents of support kettle to main kettle.

Prepare premix A (sequence 7) by adding 3% Butylene glycol to a vessel, heating to 70° C., then adding the following ingredients, one at a time: 0.2% Phenoxetol, 0.3% Barguard cp, 1% Carbowax 30, 0.25% Salicylic acid, 0.05% Clary sage, and 0.1% Resveratrol, and mixing at 2000 RPM.
Sequence 7: Add premix A to main kettle.

Prepare premix B (sequence 8) by combining 1% deionized water and 0.2% Tris amino ultra in a vessel.
Sequence 8: Add premix B to main kettle.
Sequence 9: Add 1.5% Simulgel 600 to main kettle and mix until uniform.

Formula 8 was prepared as follows:

Prepare premix B (sequence 11) by introducing 3% Butylene glycol to a vessel and heating at 70° C., followed by mixing at 150 RPM, adding 0.3% Barguard CP, then adding 0.1% Tetrahydrocurcuminoids.

Prepare premix C (sequence 12) by introducing 3% Butylene glycol to a vessel, adding 1% Carobowax, heating to 70° C., mixing at 2000 RPM, adding 0.18% Nivitol UP-302, then adding 0.1% Viapure licorice, and then adding 0.03% Viapure Citrus.

Prepare premix D (sequence 13) by introducing 4% deionized water to a vessel, mixing at 150 RPM, adding 1.8% AS-G powder and then adding 0.645% Tris amino ultra.
Sequence 1: Add 10% Hyaluronic acid to support kettle and side sweep on 15 RPM.
Sequence 2: Add 0.5% Tween 20 to support kettle.
Sequence 3: Add 23% Gransil TMG-5 to support kettle and mix at 2500 RPM.
add 10% Gransil IDS to support kettle.
add 5% DM-fluid to support kettle.
add 2% Permethyl to support kettle.
adjust temperature to 25° C.
Sequence 4: Add 1.5% KF-6017 to support kettle and mix until uniform and smooth.
Sequence 5: Add 17.46% deionized water (53-0061) to main kettle,
heat to 30° C.
when temperature reaches 30° C., mix at 530 RPM, then side sweep at 12 RPM,
add 0.2% Caffeine to main kettle,
add 0.05% EDETA to main kettle,
add 0.1% sodium ribonucleic acid to main kettle,
turn heat off, and cool to 25° C.
Sequence 6: When temperature of main kettle reaches 25° C., add 5% Yeast extract XP to main kettle,
add 2% yeast extract NAG to main kettle,
add 0.5% Rice defense complex to main kettle,
add 1% Phytoclar to main kettle, and
add 0.5% bio-converted white birch to main kettle.
Sequence 7: Add 2% Trametes extract to main kettle, mix at 1800 RPM until uniform, then side sweep at 10 RPM.
Sequence 8: Add 0.6% Aristoflex to main kettle.
Sequence 9: Add 0.2% Phenoxetol to main kettle, then add contents of support kettle to main kettle.
Sequence 10: Add 0.2% Vitamin E to main kettle.
Sequence 11: Add premix B to main kettle.
Sequence 12: Add premix C to main kettle.
Sequence 13: Add premix D to main kettle, mix at 830 RPM until uniform, then side sweep at 12 RPM.
Sequence 14: Add 2.5% Catezomes to main kettle.
Sequence 15: Add 1.5% Simulgel 600 to main kettle.
Sequence 16: Add 0.026% FD&C Yellow no. 5 to main kettle, then add 0.009% FD&C Yellow no. 6 to main kettle.

Forty-six volunteers were enrolled in a four month clinical study. Only panelists with clear visible age spots on their hands were permitted to participate. The mean age of the panelists was 62 years with the minimum and maximum ages being 44 and 82, respectively. All volunteers were Caucasian and exhibited Fitzpatrick skin type II or III. The test area was a solar lentigo on the dorsal side of each of both hands. Panelists with any kind of dermatological disorder or hypersensitivity or allergy to ethanol and/or cosmetic products were excluded from the study. Written informed consent was obtained from each volunteer before entrance into the study.

The first treatment group included 14 panelists (two age spots/panelist for a total of 28 age spots). Each panelist self-treated one age spot on each hand with a vehicle formulation, Formula 6, which served as a control. The second group of 16 panelists (two age spots/panelist for a total of 32 age spots) self-treated one age spot on each hand with a differentiation stimulating formula, Formula 7. For the third group, two formulations were provided: the differentiation stimulating formula, Formula 7, and a whitening formula, Formula 8. The 16 volunteers in this group (two age spots/panelist for a total of 32 age spots) were asked to apply the differentiation stimulating formula on one age spot on each hand, and then, after five minutes, to apply the whitening formula on top of the differentiation stimulating formula. In all three groups, panelists applied the formulation(s) twice a day for 4 months. Each panelist self-treated both hands. The panelists applied the formulation liberally on the entire dorsal surface of the hand, twice a day for 4 months: in the morning and the evening. Panelists were instructed to not use any other cosmetic products on their hands during the clinical study. On each hand, one age spot was selected by the investigator, and that one age spot was evaluated monthly. At the beginning of the clinical study (i.e., prior to treatment) and after 1, 2, 3 and 4 months, dermoscopic pictures were taken of the age spots with a dermoscopic camera (Dermalite Pro II connected to Olympus SP-510UZ digital camera). With a Dermalite Pro II dual-polarization dermatoscope, pictures of the age spots were taken in cross-polarized mode. Cross-polarized light emphasizes skin's deeper pigmentation.

These pictures were used for the calculation of contrast between the hyperpigmented spot and the surrounding skin. The pictures were transformed into black and white pictures from which grey values were calculated. Color contrast, defined as the grey value of the surrounding skin minus the grey value at the age spot, was calculated for each spot.

The hyperpigmented spots were also evaluated by in vivo RCM with the Vivascope (Lucid 1500) to allow real-time visualization of the skin in its native state. The analysis was concentrated on the confocal images taken at the basement membrane. From these images, the number of dermal papillae per mm² was counted and the circularity index of the hyperrefractive dermal papillae was calculated. The circularity index is a shape factor that refers to the alignment pattern of hyperrefractive basal cells to a circle. According to this equation, the circularity index of a perfect circle equals 100; a lower index corresponds to a more pronounced deviation from a circle and indicates a deformation of the alignment pattern of hyperrefractive cells such as commonly seen in solar lentigines. The thickness of the epidermis was also calculated.

After 1 and 4 months treatment, skin auto-fluorescence (i.e., skin's endogenous fluorescence) was measured using the Skinskan (Horiba) in order to evaluate epidermal proliferation (λₑₓ/λₑₘ=295/345 nm; reflectance 350-700 nm). The epidermal proliferation data were corrected with the corresponding reflectance data measured at 350 nm. Using fluorescence spectroscopy, the effect of varying excitation and emission can be examined. Some in vivo fluorescence bands can be attributed to specific amino acids or protein modifications. The fluorescence band attributed to tryptophan (λex/λem=295/345 nm) had been proposed as a marker for epidermal proliferation or cell renewal.

Time dependent effects were evaluated using repeated measures ANOVA calculations. Significant differences between time-points were additionally determined by a post hoc Tuckey test for multiple comparisons. The Vivascope data were statistically evaluated using a paired Student's t test.

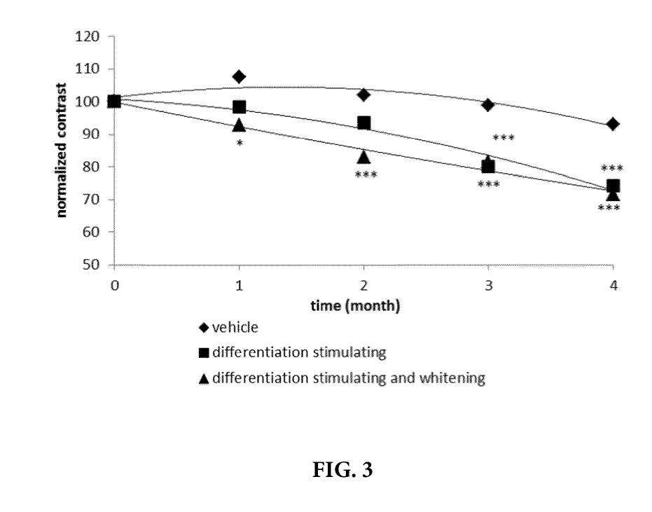

The results are graphically represented in FIGS. 3, 4A, 4B, 5, 6-8, 9A, 9B and 10.

FIG. 3 represents the skin color contrast data between the solar lentigo and surrounding skin obtained as a result of the treatments. Treatment with the differentiation stimulating formulation resulted in decreases in contrast of 2% (p=0.96), 6% (p=0.24), 20% (p<10⁻⁴) and 26% (p<10⁻⁴) after 1, 2, 3 and 4 months, respectively, compared with untreated skin. When the differentiation stimulating treatment was combined with the whitening treatment a statistically significant decrease was already observed after 1 month treatment. This combination treatment resulted in decreases in contrast of 7% (p=0.02), 17% (p=0.0001), 19% (p=0.0001) and 28% (p=0.0001) after 1, 2, 3 and 4 months, respectively. No changes in contrast were observed over the same period of time as a result of treatment with the vehicle formulation.

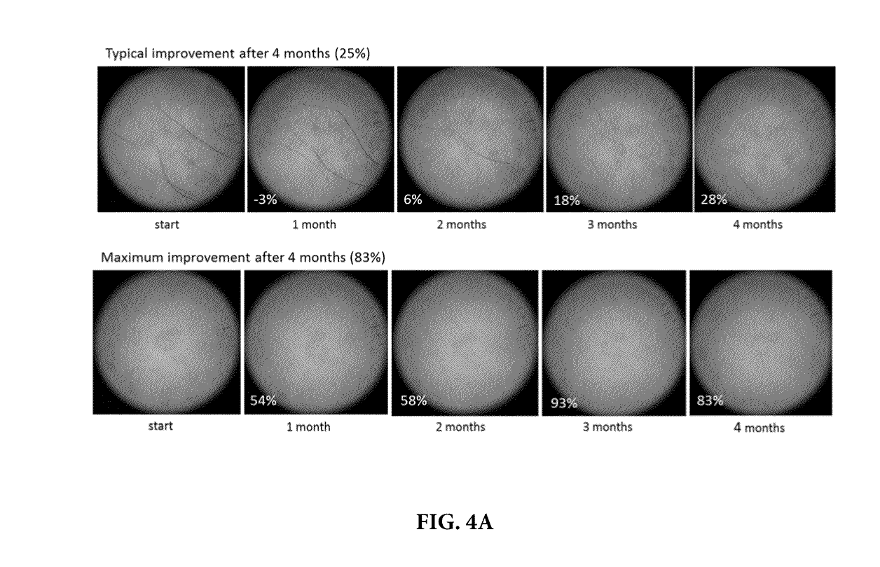

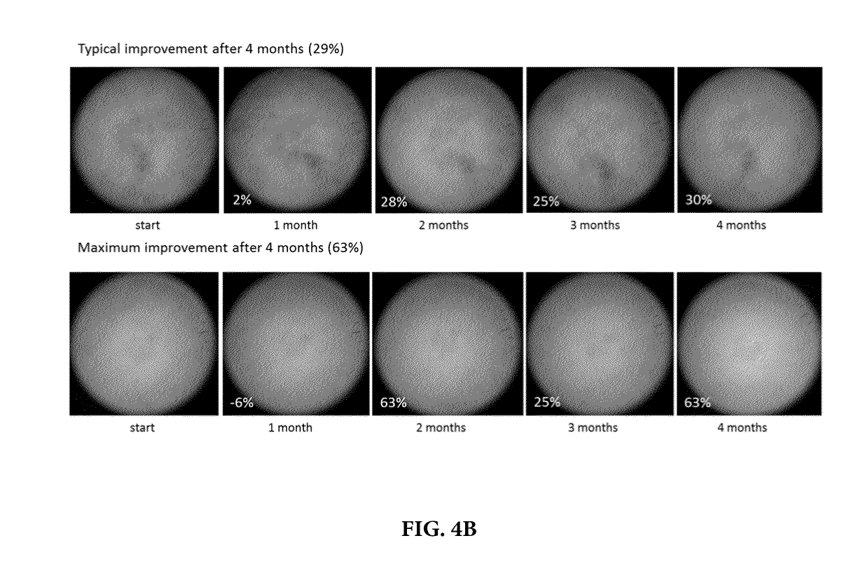

Dermoscopic pictures of solar lentigines treated with the differentiation stimulating formula are shown in FIG. 4A. The set on top shows the typical improvement in color contrast after treatment, the bottom show the maximum improvement that was found in the current test set-up.

Dermoscopic pictures of solar lentigines treated with the differentiation stimulation formula followed by the whitening formulation are shown in FIG. 4B. The set on top shows the typical improvement in color contrast after treatment, the bottom show the maximum improvement that was found in the current test set-up. For both treatments, one example is provided which shows the average improvement and another example is provided which demonstrates the maximum improvement. For the combination treatment, significance was already reached after 1 month treatment, so there was a faster decrease in color contrast as compared with treatment with the differentiation stimulation formula alone.

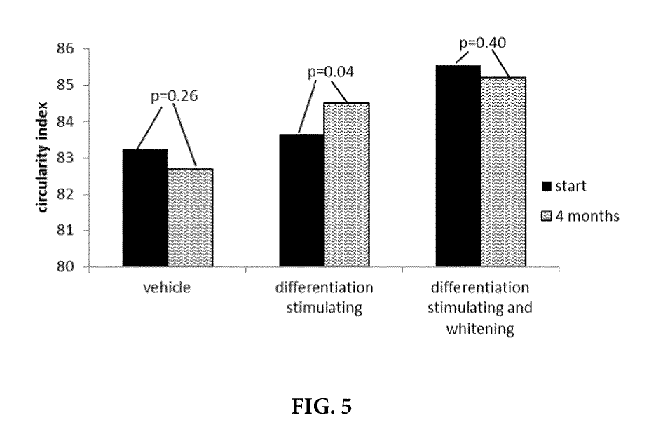

The morphology of the dermal papillae in solar lentigines (i.e., the circularity index and the number of dermal papillary rings) was investigated using RCM (Vivascope). The results for the circularity index are given in FIG. 5. After four months treatment with the differentiation stimulating formulation, a statistically significant increase in the circularity index was observed which indicated an improvement of the basement membrane. As the circularity index of a perfect circle equals 100, a lower index corresponds to a more pronounced deviation from a circle. The circularity index of the dermal papillae of non-lesional skin was found to be around 92 (data not shown).

For the vehicle and the combination treatment groups, no changes in circularity index of the dermal papillae were seen after 4 months treatment.

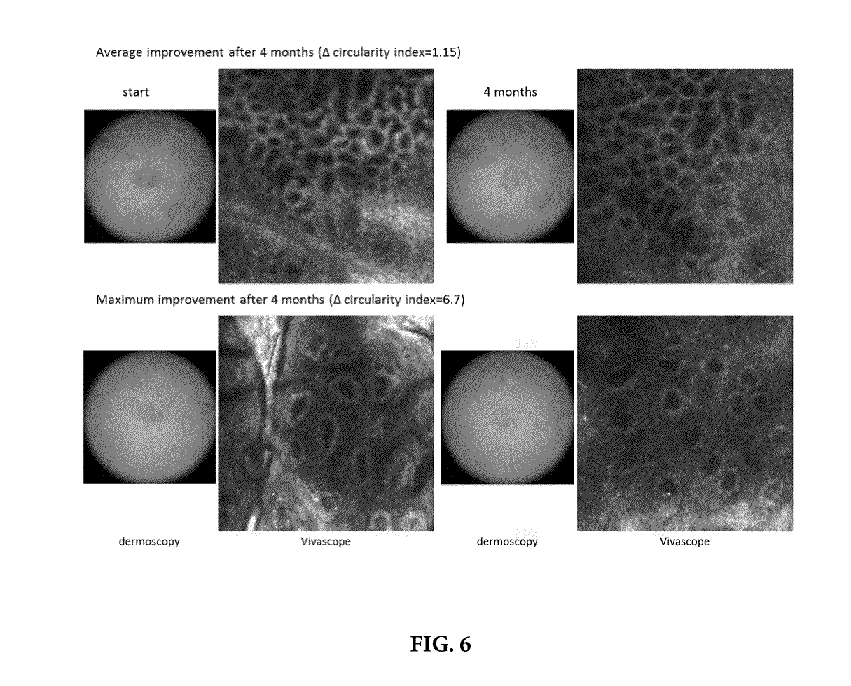

FIG. 6 depicts examples of RCM (Vivascope) images recorded at dermal-epidermal interface of solar lentigines. These images represent the average and maximum improvement after 4 months treatment with the differentiation stimulating formulation.

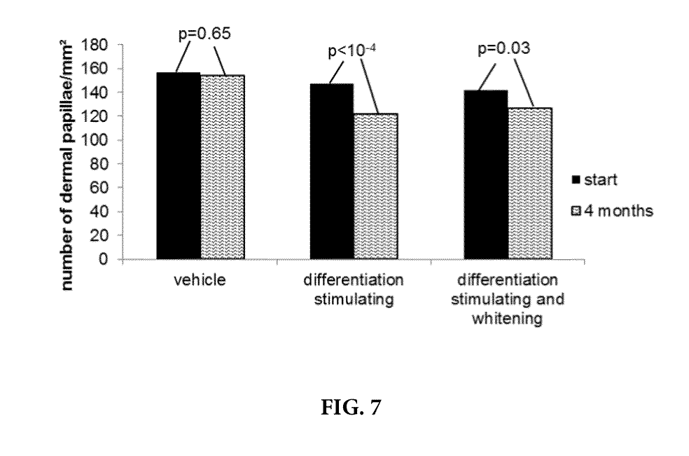

As shown in FIG. 7, a statistically significant decrease in the number of dermal papillae after 4 months treatment, compared with the pre-treatment level, was observed in the group treated with the formulation containing the differentiation stimulating actives as well as in the group treated with both the formulation containing the differentiation stimulating actives and the whitening formulation. The number of dermal papillae in non-lesional skin was observed at about 20 papillae per mm² (not shown).

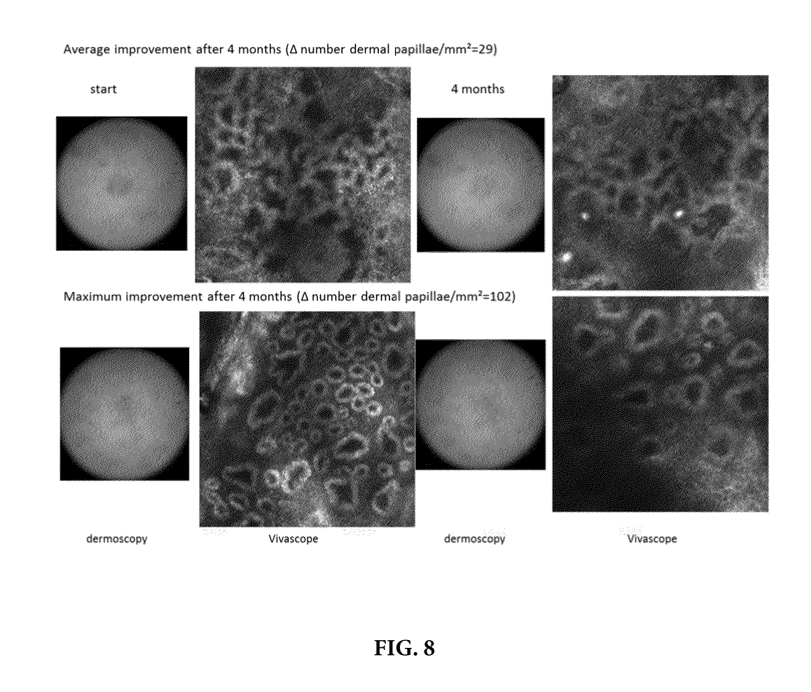

RCM (Vivascope) images taken at the dermal-epidermal interface, as shown in FIG. 8, represent the average and maximum decreases in papillary rings after treatment for four months with the differentiation stimulating formula.

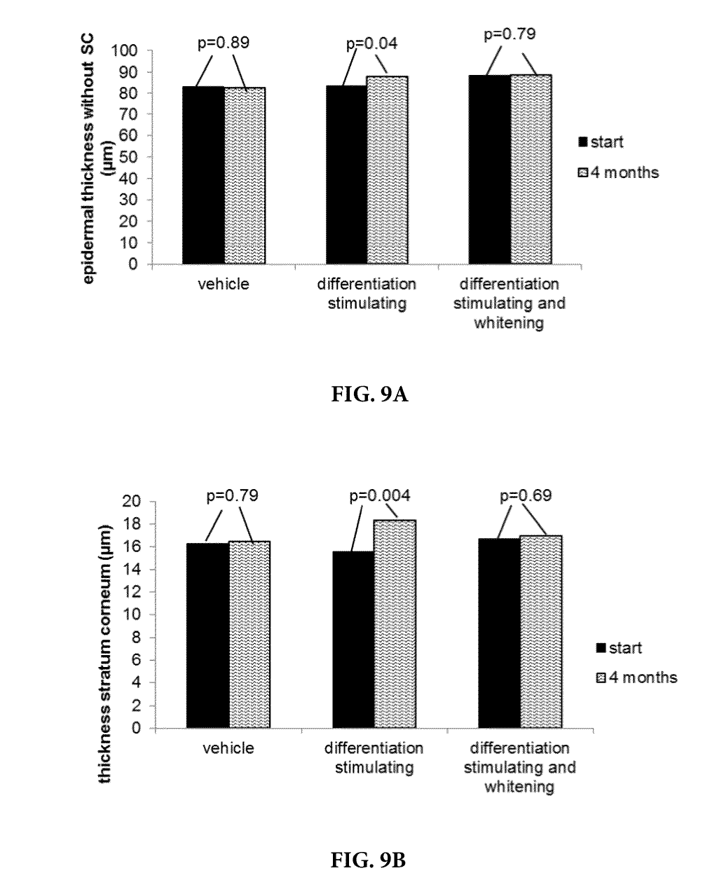

The recorded Vivascope images were further used for the quantification of the epidermal thickness. As shown in FIG. 9A, after four months of treatment with the differentiation stimulating formula, a statistically significant increase in epidermal thickness (stratum corneum excluded) was observed. In addition, a thickening of the stratum corneum was found as indicated in FIG. 9B. No differences were found after treatment with the vehicle or the combination treatment.

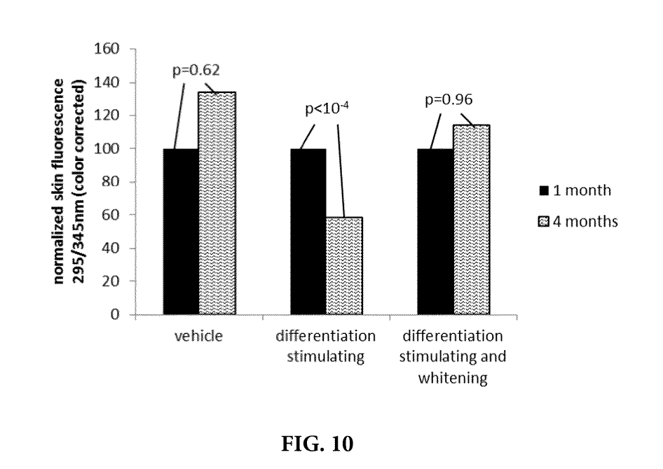

Skin auto-fluorescence (measured at 295/345 nm) results are shown in FIG. 10. The signal at these wavelengths is associated with tryptophan, a marker for the epidermal proliferation. Treatment with the differentiation stimulating formula resulted in a statistically significant decrease in epidermal proliferation after 4 months. No changes were found with the other treatments.

Treatment with the differentiation stimulating formulation over four months resulted in improved the visible appearance and morphology of solar lentigines. When a whitening formulation containing classic whitening ingredients was applied 5 minutes after the application of the differentiation stimulating formula a similar improvement in skin tone was noticed after 4 months treatment. Nevertheless, the combination treatment was somewhat less effective in improving the basement membrane compared with use of the differentiation stimulating formula alone. Although fewer dermal papillary rings were counted, no improvement could be detected in circularity index.

What is claimed is:

1. A method of identifying a material having an efficacy for reducing color contrast between a hyperpigmented skin lesion and skin surrounding the hyperpigmented skin lesion without directly affecting melanocytes or melanogenesis, said method comprising:
(a) applying a composition containing a material to be tested to the hyperpigmented skin lesion; and
(b) after an interval of time, observing whether the composition has effected at least one of inhibiting proliferation of keratinocytes, stimulating differentiation of keratinocytes, and improving compressive deformation of dermal papillae, in a basal layer of epidermis in the hyperpigmented lesion of skin.

2. The method of claim 1, wherein the interval of time is at least about one month.

3. The method of claim 1, wherein the interval of time is at least about two months.

4. The method of claim 1, wherein the interval of time is at least about three months.

5. The method of claim 1, wherein the interval of time is at least about four months.

6. The method of claim 1, wherein the effect of the composition on inhibiting proliferation of keratinocytes is evaluated using Reflectance Confocal Microscopy (RCM).

7. The method of claim 1, wherein the effect of the composition on stimulating differentiation of keratinocytes is evaluated using Reflectance Confocal Microscopy (RCM).

8. The method of claim 1, wherein the effect of the composition on improvement in compressive deformation of dermal papillae is evaluated using Reflectance Confocal Microscopy (RCM).

9. A method for lightening skin, comprising applying to skin in need of said lightening, a topical composition comprising at least one active which effects at least one of inhibiting proliferation of keratinocytes, stimulating differentiation of keratinocytes, and improving compressive deformation of dermal papillae in a basal layer of epidermis in a hyperpigmented lesion of skin, wherein the active enables lightening of the skin in a hyperpigmented lesion relative to skin surrounding the hyperpigmented lesion without directly affecting melanocytes or melanogenesis.

10. The method of claim 9, wherein the topical composition further comprises a skin lightening active which directly affects melanocytes or melanogenesis.

11. An improved topical cosmetic composition for reducing color contrast between a hyperpigmented skin lesion and skin surrounding the hyperpigmented lesion, said composition comprising at least one first active which directly affects melanocytes or melanogenesis, in a cosmetically acceptable vehicle therefor, wherein the improvement comprises including in said composition a second active for effecting at least one of inhibiting proliferation of keratinocytes, stimulating differentiation of keratinocytes, and decreasing compressive deformation of dermal papillae in a basal layer of epidermis in the hyperpigmented lesion of the skin, without directly affecting melanocytes or melanogenesis.

12. A regimen for reducing color contrast between a hyperpigmented lesion of skin and skin surrounding the hyperpigmented lesion, comprising:
(a) applying to skin in need of such reduction in color contrast, a first cosmetic composition comprising a first skin lightening active for at least one of inhibiting proliferation of keratinocytes, stimulating differentiation of keratinocytes, and improving compressive deformation of dermal papillae in a basal layer of epidermis in a hyperpigmented lesion of skin, without directly affecting melanocytes or melanogenesis, in a cosmetically acceptable vehicle therefor; and
(b) applying to the skin in need of such reduction in color contrast a second cosmetic composition comprising a second skin lightening active which directly affects melanocytes or melanogenesis, in a cosmetically acceptable vehicle therefor; wherein (a) and (b) may occur in any order.
